(19) United States
(12) Patent Application Publication
(10) Pub. No.: US 2002/0132227 A1
(43) Pub. Date: Sep. 19, 2002

(54) Approaches for HPV detection and staging by targeting the E6 gene region of the viral genome

(75) Inventor: Eugen Molodysky, (US)

Correspondence Address: LADAS & PARRY, 26 WEST 61ST STREET, NEW YORK, NY, 10023, US

(73) Assignee: BIOSEARCH INTERNATIONAL PTY. LTD.

(21) Appl. No.: 09/818,982

(22) Filed: Mar. 27, 2001

Related U.S. Application Data
(60) Provisional application No. 60/192,673, filed on Mar. 28, 2000.

(51) Int. Cl. C12Q 1/70; C12Q 1/68; C12P 19/34

(52) U.S. Cl. 435/5; 435/6; 435/91.2

(57) ABSTRACT

The L1/E1 gene region of the HPV virus maybe deleted during integration into the genome of the host cell, but the E6/E7 gene region is always retained. There is a need to detect HPV infection and cervical cancer in way that provides information about the stage of infection so that the proper treatment can be undertaken.

### FIELD OF THE INVENTION

Integration of the E6 region of human papillomavirus into the cellular DNA is an important step in the progression to malignancy. This invention involves methods for detecting human papillomavirus in cervical cells and determining the progression of the infection.

### BACKGROUND OF THE INVENTION

Infection by HPV involves the passage of the viral DNA into a cell. The HPV viral genome can be divided into 3 regions, upstream regulatory region (URR) or long control region (LCR), the early gene region and the late gene region. These regions control sequences for HPV replication and gene expression, encoding the E2, E6 and E7 genes, and encoding the L1 and L2 genes respectively (Turek, Adv. Virus Res. 44:305-356 (1994)). Initially at least the circular HPV DNA remains free inside the cell in an episomal form. Whereas the episomal form predominates early in infection, this situation may change later, with the subsequent occurrence of integration. Although initially some episomal HPV DNA remains along with the integrated HPV DNA in an infected cell, ultimately, in a significant proportion of cancers the integrated form not only dominates, but represents the only HPV DNA present. The evidence suggests that integration may be an important step in the progression to malignancy.

The viral genomes are exclusively maintained as episomes in benign lesions induced by HPV types such as 6 and 11 (Dowhanick et al, Suppression of cellular proliferation by the papillomavirus E2 protein. J. Virol 1995; 69:7791-7799; Kobayashi et al., Presence of human papillomavirus DNA in pelvic lymph nodes can predict unexpected recurrence of cervical cancer in patients with histologically negative lymph nodes. Clin. Cancer Res 1998; 4:979-83). Only episomal HPV is detected in CIN I and integrated sequences are rarely found in CIN II and CIN III (Cullen et al. Analysis of the physical state of different human papillomavirus DNAs in intraepithelial and invasive cervical neoplasms. J. Virol, 1991; 65:606-612; Das et al. “Analysis by polymerase chain reaction of the physical state of human papillomavirus type 16 DNA in cervical preneoplastic and neoplastic lesions. J. Gen. Virol. 1992; 73:2327-2336). In contrast, the viral DNA is usually integrated into the cellular genome in cell lines derived from cervical carcinomas (Boshart et al., “A New Type of papillomavirus DNA, its presence in genital cancer biopsies and in cell lines derived from cervical cancer. EMBO J. 1984 3:1151-1157; Howley, P. M. Presence and expression of human papillomavirus sequences in human cervical carcinoma cell lines. Am J. Pathol 1985 119:361-366; and Tsunokawa et al., “Presence of human papillomavirus type-16 and type-18 DNA sequences and their expression in cervical cancers and cell lines from Japanese patients. Int. J. Cancer 1986:37:499-503; and Yee et al.”Presence and expression of human papillomavirus sequences in human cervical carcinoma cell lines. Am. J. Pathol. 1985: 119:361-6) . This suggests that integration begins early in cancer development and is an important event in malignant transformation (Bosch et al. “Prevalence of human papillomavirus in cervical cancer: a worldwide perspective. J. National Cancer Inst. 1995; 87:796-802; Cullen, et al. Analysis of the physical state of different human papillomavirus DNAs in intraepithelial and invasive cervical neoplasms. J. Virol 1991; 65:606-612; and Vernon et al. “Association of human papillomavirus type 16 integration in the E2 gene with poor disease-free survival from cervical cancer. Int. J. Cancer 1997: 74:50-56). Thus the accurate detection of the L1 versus E6 status of the HPV DNA may be very important in determining cervical cancer progression and in assisting in clinical management of those women.

Integration was first described by the Heidelberg group of zur Hausen and Gissman (Dürst et al.J. Gen. Virol. 1985, 66 (Pt 7):1515-22; Schwarz et al. Nature 1985; 314:111-4). For HPV 16, cancer cells displayed both integrated and episomal forms, with the integration occurring as head-to-tail arrays (Dürst et al. J. Gen. Virol. 1985, 66 (Pt 7) 1515-22). In the case of HPV18, Schwarz et al. Nature 1985, 314:111-4) found that in 3 cell lines, the HPV DNA was integrated and amplified. A mixture of integrated and episomal HPV16 in carcinomas was also found by Fukushima et al. Cancer 1990, 66:2155-61). Yiu et al. Oncogene 1991; 6:1339-42, noted that of 9/15 cervical carcinomas with HPV16 DNA, 44% had integrated, 44% had episomal and 11% both. Matsukura et al. Virology 1989: 172:63-72 (1989) found both integrated and episomal HPV16 in 34 invasive cervical cancers. Eight of these exhibited only the integrated form of HPV DNA.

In a study of cervical cancers by Park et al. Gynecol. Oncol. 1997; 9:267-76, HPV16 or 18 DNA was found to predominate, being present in 75% of 68 cases, with 7% having episomal only, and 18% a mixture of each. It was apparent to these workers that a difference existed between HPV 16 and HPV 18 in that, of the 51 with HPV 16, 71% had only integrated HPV DNA, 20% had both integrated and episomal and 10% episomal only. In contrast all 17 HPV 18-containing cancers revealed only integrated HPV DNA. This is a significant observation, given claims of the aggressive, rapidly progressing nature of HPV 18-associated dysplasias, and is highly relevant to screening tests.

An overview of the various published studies by Pfister & Fuchs, Dermatol. Clin. 1991; 9:267-76 concluded that 36-71% of cervical cancers had just episomal HPV DNA, cancers in which the HPV DNA present was completely in the integrated form were 22-39% of the total, and those with both episomal and integrated HPV DNA comprised 6-25%.

Early observations for dysplasias with HPV16 infection showed evidence of integration in 86%, i.e, integration occurs in the precancerous stage of HPV's clinical course (Shirasawa et al. J. Gen. Virol 1986, 67:2011-5).

Fukushima et al. Cancer 1990; 66:2155-61 looked at CIN samples as well and found that of 7 positive for HPV16, 3 contained only integrated HPV16 DNA.

In a study of different stages of the carcinogenic process by Cullen et al. J. Virol. 1991; 65:606-612 it is stated that integration is a characteristic of malignant lesions. Of 100 CIN biopsy specimens they found 3% with integrated HPV. However, for 69 carcinomas, 81% displayed integration. The most common HPV was type 16 (40/69=58%). Of these, 72% contained integrated HPV DNA and for 27% the HPV16 DNA was exclusively episomal. For the specimens that had integrated HPV DNA, in 80% of cases this was the only form in which the viral DNA was present in the cell. Only 20% had episomal as well as integrated HPV DNA.

Cullen et al J. Virol. 1991; 65:606-612 also found in the case of HPV18, which has greater transforming efficiency that all 23 carcinomas had integrated HPV DNA, with only 1 having episomal DNA as well.

In the early work by Schwarz et al. Nature 1985;324: 111-4 deletion of up to 2-3 kb of DNA was found in cell lines containing HPV DNA. This included the E2 to L2 region. Importantly, E6-E7 transcripts could be detected in these cells.

Subsequent work by Shirasawa et al. J. Gen. Virol., 68:583-91, 1987) on cervical carcinoma cell lines found that open reading frames (ORFs) El, E2, E4 and E5 were interrupted by flanking host cell DNA suggesting that the integration into host cell DNA was occurring preferentially in these regions of the HPV genome. They found that HPV mRNAs hybridized with the entire E6 and E7 ORF and a minor part of the E1 ORF, meaning that these were the only portions of the HPV genome present. No hybridization to L1 and L2 ORFs could be detected, implying that these regions of the virus had been deleted.

In a study of 6 cervical carcinoma samples Choo et al. (Virol. 1987: 161:259-61 confirmed that on integration, the E6/E7 region is retained, but found that the E2 region is lost.

Matsukura et al J. Virol. 198658:979-82 found most of E1 and all of E2 to be deleted in a cervical carcinoma containing HPV16 DNA. Interestingly, in one study of a cell line, the integration that disrupted E2 and L2 was found to have occurred in the premalignant lesion from which the line was derived (Schneider-Maunoury et al. 1987, J. Virol : 61:3295-8). Wagatsuma et al. J. Virol (1990) 64:813-21) found 4 clones of integrated HPV16 DNA that had deletions in the E1/E2 and the L1/L2 regions, and they state that no site specific for integration is present in the viral sequence. Rather, during integration, viral sequences are opened within any ORFs except the E6/E7 ORFs and locus control region. Jeon et al. J. Virol. 1995; 69:2989-97 showed that integration of the viral genome into the human chromosome in the cancer cells usually disrupts or deletes the E2 ORF, which results in the loss of expression of the E2 gene. This was associated with the expression of high levels of E6 and E7 being maintained (Jeon et al., J. Virol 1995; 69:2989-97).

Mutations of HPV DNA take place in the L1 region of the HPV genome (but never the E6 region). In a detailed analysis of DNA from an HPV16-positive cervical carcinoma only 3091 bp of the original 7905 bp viral genome remained (Cone et al. J. Med. Virol1992 37:99-107). This included the E6/E7 region. Moreover, whereas the E6 and E7 ORFs showed complete concordance with the published sequences (which also supports the role of E6/E7 in tumorigenesis), there were multiple mutations (transversions, transitions, small deletions, and small insertions) in the remaining integrated HPV 16 DNA, which was composed of parts of the L1 and E1 ORFs. (The 5′ end of L1 was missing, the integrated DNA beginning at nt 6334). The mutations included: a single base deletion at nt 6387; insertion of nucleotides CAT at position 6901 (which has also been noted by others, viz. Baker et al. J. Virol1987 61:692-71; Choo et al., J. Virol 1988 62:1659-66; Matsukura et al. J. Virol. 1986; 58:979-82); and deletion of GAT at nt 6949.

Integration of HPV16 DNA has in fact been found to lead to increased steady state mRNA encoding the viral oncogenes E6 and E7 as a consequence of increased stability conferred by disruption of A+U rich sequence in 3′-UTR of E6 and E7 mRNAs and replacement with cellular sequences with lower A+U content (Jeon & Lambert, J. Virol. 1995; 69:2989-97). This would account at least in part for the higher concentration of E6 and E7 proteins in clonal populations with integrated HPV16 DNA compared with ones in which the DNA is present exclusively in an episomal form (Jeon et al. J. Virol.1995; 69:2989-97). The cells with integrated DNA therefore outgrow those with episomal HPV DNA only, i.e. integration provides a growth advantage (Jeon et al. J. Virol. 1995; 69:2989-97).

It is widely known that the E6 product binds p53, an important negative regulator of the cell cycle, and in so doing inhibits its activity, so leading to uncontrolled growth. This offers a biological basis for oncogenesis. Similarly the E7 protein attaches to another crucial cell cycle regulator, the retinoblastoma binding protein. It is also known that the E2 gene encodes a site-specific DNA-binding protein that is involved in the regulation of the HPV promoter that directs E6 and E7 expression (Romanczuk et al. J. Virol, 1990; 64(6):5240-9; Thierry & Howley, 1991 New Biol; 3:90-100; Bernard ct al., J. Virol.1989; 63:4317-4324).

As far as cervical cancer is concerned what matters is the E6 and E7 region. The rest of the genome may have had a role in transmission of the virus to a new host cell, but it is the proteins encoded by E6 and E7 that cause the cancer. Integration of HPV DNA occurs early in cancer development and is important as an activation mechanism for progression from precancer lesions to cancer lesions (Bosch et al. J. Natl. Cancer Inst. 1995; 87:796-802; Cullen et al. J. Virol 1991 65:606-612; Vernon et al. Int. J. Cancer 1997:74-50-56).

Targeting the available E6 region rather than the deleted L1 or L2 regions would appear to be the best way to ensure that the critical DNA in cancer causation is not missed. This is consistent with the fact that the L1 and L2 regions of the HPV genome can be deleted during integration into the genome of the host cell, but the E6/E7 region is always retained. Approaches targeting the L1 and L2 regions therefore would appear to be inferior. Since deletion or mutation can involve specimens at an advanced stage of abnormality, the resulting false negative result could have fatal consequences. In contrast, primers that detect high risk types of HPV by the targeting the E6 region should not miss one of these high risk HPV infections. It is just a matter of ensuring that the relevant primers are included in the E6 PCR test that is performed.

A popular approach for detecting HPV in a cell involves using primers directed at the L1 region of the virus. See U.S. Pat. Nos. 5,182,377; 5,283,171 and 5,447,839. However the approach of directing primers at the L1 region has serious disadvantages and directing the primers at the E6 region is preferable in the clinical setting. Integration can occur in the L1 region but not the E6 region.

Another strategy involves PCR targeted to the cancer-causing E6 part of the virus (Morris & Nightingale 1987; Morris et al., 1988, 1990; and Dallas et al., 1989) because this region shows the greatest differences in nucleotide sequence between different HPV types. This makes it possible to design type- or clade-specific primers in order to not only determine if HPV is present, but at the same time distinguish high- from low-risk HPV types. HPV groupings based on the sequence homology between the E6 regions of HPVs correlate with clinical significance (Lorinez et al. 1992), leading to support for risk-group specific primers. The value of this approach has been confirmed (Fujinaga et al. 1991) and offers a direct means of getting the kind of information that is needed for clinical decision making. It is likely that all of the major HPV types are now known, so that suitable primers directed at the E6 region of the various types, and capable of telling whether a woman is infected with a high-risk HPV, can be synthesized and readily incorporated into the same reaction tube. This makes the chance of missing an infection very unlikely indeed. Costs are also lower, since fewer steps are needed. This makes E6 testing more attractive in widespread screening.

This approach formed the basis for the original PCR for HPV detection (Morris & Nightingale 1987; Morris et al. 1988, 1990; Dallas et al. 1989). The idea of targeting the PCR primers to the E6 region was based on a number of important facts, not the least of which was the well-recognized oncogenic role of E6 in cancer. Primers were designed against sequences within the E6 region that were conserved between different high risk types. Other common primers were designed that would only hybridize within the E6 region of low-risk types. Thus there were primers that would hybridize to the most common low-risk HPV types (HPV6 and HPV 11) and others that would hybridize to the most common high-risk types (HPV16 and HPV18). By ensuring that PCR products of different sizes would emanate from this choice the test could discriminate high- and low risk HPV groups. For low-risk types (HPV6 and HPV11) the size of the band seen on electrophoresis was ˜120 bp. For HPV16 (and HPV33, which could be detected using the same primers) it was ˜200 bp and for HPV18 it was ˜100 bp. Confirmation of type was made by type-specific primers, although these were not necessary for routine screening. For the residue of rarer HPV types not covered by the test developed originally, other primers could be readily designed using the same principal, and added into the mixture of primers in the PCR.

As stated, there is evidence that support the concept that integration of the HPV viral genome into the human chromosome may delete or disrupt the L1 and other gene regions, whilst maintaining high levels of E6 and E7 expression (Armstrong & Holman, 1981; Figge et al. 1970). This deletion or disruption results in the loss of expression of the L1 gene region, causing an absence of the L1 gene region sequences following integration. Thus, though the targeting of the E1 or L1 region has the potential to detect many, perhaps all, HPV types detection will not occur if the virus is present in an exclusively integrated form. It is known that the target for PCR will not always be retained if it is in L1 and other regions besides E6 and E7. On the other hand targeting the E6 or E7 regions will detect all HPV-positive patients, provided that primers for all relevant high-risk HPV types are used. That the method of detection strongly influences the rate of HPV detection was emphasized by Noffsinger et al. (1995). In a study of anal carcinomas they showed that the least sensitive method for HPV detection is PCR using L1 consensus primers (rate=16%). On the other hand, type-specific primers directed at the E6 region yield a positiviity of 46% in anal carcinomas.

L1 PCR probably detects the majority of cases with HPV types 6 or 11, but underestimates HPV16 infections. A suggestion that this is because of fragmentation of template DNA in formalin-fixed tissues (Park et al., 1991) is unlikely, as others have consistently been able to amplify HPV DNA from such specimens (Noffsinger et al. 1995). Even though L1 PCR products can be 450 bp, type-specific primers give 380-440 bp PCR products from formalin-fixed speciments. The more likely explanation was said to be loss of HPV genomes upon integration, in particular deletion of L1 and L2 (Noffsinger et al. 1995). Transcripts from these regions are indeed lost in HPV 16 infected cells (Stoler et al, 1992). The loss of late region genes is further supported by the rarity of positivity for viral capsid proteins in intra-epithelial and invasive anogenital neoplasia (Schwarz et al., 1985; Durst et al., 1989). Thus PCR strategies that rely on the presence of late genes may significantly underestimate the number of cases that are HPV positive. In contrast, the E6 and E7 genes are highly conserved (Cone et al., 1992; Stoler e al., 1992, Baker et al., 1987; Wagatsuma et al., 1990) and Noffsinger et al., (1995) state that HPV infection should therefore be detectable in almost all cases where viral DNA is present.

It is known in the art to target so-called “consensus” primers to the highly conserved L1 region of the HPV genome. An extra step is required such as probing with radioactive- or biotin-labelled type-specific oligonucleotides in order to address the important question of type of HPV present.

Consensus primer sets include those directed at various parts of the L1 region, viz. ones that have been termed by those who developed them My09-My11, Gp5-Gp6, Gp5+-Gp6+, and oli-lb-oli-2I, and those directed at the E1 region, viz-CpI-CpIIG (see Karlsen et al. 1996 for an overview). The MY09/MY07 (MY-PCR) primer set (Manos et al. 1989) and the GP5+/GP6+(GP+-PCR) primer set (de Roda Husman et al. 1995) are currently the most commonly used primer sets for HPV detection in clinical samples. The latter are a modification (extension in length) of an earlier version, GP5/GP6, which were designed to amplify the nt 6624-6765 region of the HPV genome to yield a 140-150 bp product (Snijders et al. 1990). The MY-PCR set amplify the nt 6582-7033 region to yield a 450 bp product (Manos et al. 1989). The MY-PCR is used more in America and Asia, and the GP+-PCR in Europe, reflecting the geographical locality where each set was developed. The MY-P CR primer set is synthesized with several degenerate nucleotides in each primer and is thus a mixture of 25 primers capable of amplifying a wide spectrum of HPV types (Manos et al. 1989; Hildesheim et al. 1994). In contrast, there are only two primers in the GP+PCR set and detection of a broad range of HPVs is achieved by using a lowered annealing temperature during PCR (de Roda Husman et al. 1995). For E1, two 21 mers have been described that amplify within this conserved region of HPVs tested (Gregoire et al., 1999).

In a comprehensive study that addresses this issue, Karlsen et al. (1996) used a range of consensus primer sets, including those directed at various parts of the L1 region, viz. My09-My11, Gp5-Gp6, Gp5+-Gp6+ and oli-lb-oli-2I and those directed at the E1 region, viz CpI-CpIIG. By testing with all of these primer sets, as well as primers directed at the E6 or E7 region, 98% of 355 biopsy specimens from patients with invasive cervical carcinomas were found to be positive for HPV. However, use of just one gave a much lower rate of detection. It is interesting to note that type-specific primers (for HPV11, 16, 18, 31, 33 and 35) detected more HPV-infected patients than the most sensitive consensus primer set. In fact it was necessary to use several consensus primer sets together (viz. The My/Gp/Gp+ and Cp sets) in order to detect a high number of HPV-positive patients. Moreover, based on results using consensus primers, L1 deletions were present in 23 of 56 (41%) samples. The data argued strongly against the reliability of using L1 consensus primers alone. Deletions mean not all consensus primers will have hybridization targets in the HPV DNA leading to the conclusion that a combination of consensus primers must be included in any PCR test to have any hope of detecting all or more HPV present in a specimen (Karlsen et al. 1996).There appears to be agreement that use of either the commonly used MY-PCR or GP+-PCR methods alone will underestimate the true prevalence of HPV in cervical samples (Smits et al. 1995; Karlsen et al. 1996).

In other studies, involving 635 cervical cancer samples from Spain, Colombia, and Brazil (i.e. 436 in a report by Munoz et al., 1992 and 199 cases in a study by Eluf-Neto et al., 1994), HPV was detected in just 85% of samples using only one consensus primer set. Even lower rate of detection emerged from a study involving the MY L1 primers, which gave only 69% positivity (Guerrero et al. 1992).

Since integration is an important step in the progression to malignancy, the spectre of missing HPV during testing by PCR if the primers are directed at a mutated or variable region, even if this region is not deleted. That is, one has to worry about both deletion and mutation if targeting the L1 region using a consensus PCR. This is least likely to happen for the E6 or E7 region, since any mutation could have functional implications, most likely deleterious to viral oncogenic function so that the ramifications of failure to detect are likely to be inconsequential. In reality, there is “extraordinary conservation of the E6/E7 DNA sequence” (Cone et al., 1992) meaning that primers directed at the E6 region have a very much greater likelihood of annealing than ones targeting the L1 region. The implication of these findings is that whereas screening in women with CIN will probably pick up more HPV infections when L1 primers are used for PCR, some women with more advanced lesions may be missed because of the loss of L1 during integration. And it is these women in particular who must be correctly diagnosed, since their need for treatment is greater and has to be instituted as a matter of priority.

Approaches targeting the L1 are therefore inferior. Since deletion or mutation can involve specimens at an advanced stage of abnormality, the resulting false negative result could have fatal consequences. In contrast, methods that detect high vs low risk groups of HPV by targeting the E6 region should never miss an infection.

Thus targeting the available E6 region rather than the deleted L1 region is the best way to ensure that the critical DNA in cancer causation is not missed. Targeting the E6 or E7 region will detect all HPV-positive patients provided that all relevant high-risk HPV types are screened for.

WO 99/29890 discloses methods for monitoring the stages of HPV-induced diseases by measuring the expression levels of mRNA from the E6, E7, E2, E1 regions, and determining the ratio of the expression level of E6 and/or E7 to L1 and/or E2.

The presence or absence of a particular form of HPV DNA may be indicative of the cervical cell state. That is there is likely to exist a continuum of HPV DNA forms that exist in parallel with cervical cell disease. Specifically, an episomal HPV infection may be considered representative of early stage HPV infection of CIN I with integrated HPV infection being considered representative of cancer. The determination of the presence or absence between the two ends of this spectrum may provide an indication of the stage of HPV-based cervical disease.

Therefore, it is an object of this invention to detect the presence or absence of the E6, E7 or E6/E7 regions of HPV and the presence or absence of the L1, L2 or L1/L2 region of HPV in a sample of cervical cells.

It is a further object of this invention to use these findings to assess the risk of developing HPV-based disease and/or to determine the stage of infection.

### SUMMARY OF THE INVENTION

As the E6/E7 gene regions are retained and the L1/L2 regions are deleted in the process of disease progression, measuring of the relative amounts of DNA, RNA or expression of the E6 and/or E7 regions and L1 and/or L2 regions of HPV infected cells provide:

1. A method for assessing the stage of HPV-based disease;

2. A method for assessing the risk that a woman with HPV infection will develop HPV-based disease; and

3. A method for categorizing/staging women with HPV infection but without detectable HPV-based disease into those at risk for progression to disease and those not at risk of progression to disease.

### DETAILED DESCRIPTION OF THE INVENTION

There are methods known in the art for detecting DNA and RNA. A number of methods are particularly suited for detecting viral DNA or RNA. Any method that can be used to detect the presence or absence of the E6 and/or E7 and L1 and/or L2 regions of the HPV genome can be used in the method of this invention.

Methods for detection of HPV DNA or RNA include but are not limited to polymerase chain reaction (PCR) (See U.S. Pat. Nos. 4,683,195; 4,683,202; 4,800,159; 4,965,188; 5,008,182 5,176,995; 5,182,377; 5,283,171, and WO 88/06634); light cycla, Taq Man, Q-beta replicase, ligase chain reaction, PCR-Elisa and NASBA DNA detection.

PCR can be used with ELISA (enzyme-linked immunosorbent assay) to identify DNA sequences in the E6/E7 and L1/L2 regions of HPV.

The ligase chain reaction is a DNA amplification technique which is a cyclic two-step reaction: 1) a high-temperature melting step in which double-stranded target DNA unwinds to become single-stranded and 2) a cooling step in which two sets of adjacent, complementary oligonucleotides anneal to the single-stranded target molecules and ligate together. The products of the ligation from one cycle serve as templates for the next cycle's ligation reaction. Amplification is achieved in a similar manner to PCR. See Weiss, (1988) Science 254, 1292-1293; Landegren, U. et al. (1988) Science 241:1077-1080; Barany, F. (1991) PCR Methods and Applications 1:5-16 and Marsh, E., et al. (1992) Strategies 5:73-76.

Q-beta replicase is an isothermal nucleic acid amplification system which uses the enzyme Q-beta replicase. See U.S. Pat. Nos. 5,556,751 and 6,004,747.

Taq Man involves the utilization of an additional oligonucleotide in the standard PCR reaction. The additional oligonucleotide hybridizes to a region between the forward and reverse oligonucleotides. The additional oligonucleotide is conjugated to a fluorescence molecule on the 5′ end and a quenching molecule on the 3′ end. When these two molecules are in proximity the fluorescence is quenched. When the Taq polymerase encounters this oligonucleotide it chews it off releasing the fluor and the quencher causing a change in fluorescence. The oligonucleotide probe that is specific for the target to be amplified is labelled with a fluorescent tag and a quenching molecule. During the extension step of PCR the Taq enzyme will disrupt probe bound to the target separating the fluorescent tag from its quencher molecule thus permitting fluorescence. In another approach an oligonucleotide probe containing a reporter molecule-quencher molecule pair that specifically anneals to a region of a target polynucleotide downstream i.e. in the direction of extension of primer binding sites. The reporter molecule and quencher molecule are positioned on the probe sufficiently close to each other such that whenever the reporter molecule is excited, the energy of the excited state nonradiatively transfers to the quencher molecule where it either dissipates nonradiatively or is emitted at a different emission frequency See U.S. Pat. Nos. 5,210,015 and 6,030,787.

NASBA (Nucleic acid sequence based amplification) is an isothermal RNA amplification method using reverse transcriptase. NASBA is continuous rather than cyclic which means it measures all at once instead of waiting for a series of copies to be made. Quantitative detection is achieved by way of internal calibrators which are added at isolation and are co-amplified and subsequently identified along with the wild type of RNA using electrochemiluminscence.

These methods and others known in the art can be used to detect DNA and/or RNA and/or expression of genes of HPV.

The DNA sequences of strains of HPV are known. Known HPV include HPV 1a, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 47, 48, 49, 50, 51, 52, 53, 54, 55, 56 , 57, 58, 59, 60, 61, 62, 63, 64, 65, 66, 67, 68, 69, 70, 72, 73, 74, 75, 76, and 77. The DNA sequences of these viruses can be found in the GenBank database.

The sequences of HPV 6, 11, 16, 18, and 33, are also described in the following references.

The sequence of HPV6 is given in Schwartz et al., EMBO L 2:2361-8, 1983.

The sequence of HPV11 is given in Dartman et al. Virology 151:124-30, 1986.

The sequence of HPV16 is given in Seedorf et al. Virology 145:181-5, 1985.

The sequence of HPV18 is given in Matlashewski et al., J. Gen. Virol. 67:1909-16, 1986.

The sequence of HPV33 is given in Cole and Streeck. J. Virol 58:991-5, 1986.

The methods described above can be used to detect the presence or absence of the E6, E7 or E6/E7 region and the presence or absence of the LI, L2 or L1/L2 region. The presence or absence can be detected by using a method that detects or measures DNA, RNA or expression of the gene. The methods can also be used to determine whether the DNA, RNA or gene product is from a high risk or low risk strain of HPV. If it is determined that the DNA, RNA or gene product is derived from a high risk strain of HPV, the presence or absence of DNA, RNA or gene product from the L1 or L2 region will provide the clinician with important information about the progression of the infection. If the L1 or L2 regions are present then it is likely that the cell has not completed its transformation to the malignant state. If no L1 or L2 regions are present and only the E6, E7 or E6/E7 regions can be detected then the cell has been transformed to a malignant state and clinical intervention and further testing and treatment is warranted.

If integration occurs in the precancerous stage and this occurs in the L1 gene region, and if the E6 region is not integrated and remains detectable in the advancing stages of carcinogenesis, then combined L1/E6 PCR testing provides a diagnostic/prognostic indicator of progression.

Tables 1-3 show clinical scenarios:

1. L1 negative and E6 negative=negative HPV

2. L1 positive and E6 positive=early disease state

3. L1 negative and E6 positive=advanced disease state

TABLE 1
INTEGRATION/DELETION SITES

| | Integrated | |
|---|---|---|
| Study | Deletion Sites | Retention Sites |
| Schwartz et al (1985) | E2 to L1 | E6, E7 |
| Shirasawa et al (1987) | E1, E2, E4, E5, L1, L2 | E6, E7 |
| Choo et al (1987) | E2 | E6, E7 |
| Matsukura et al (1986) | E1, E2, L2 | ? |
| Waggatsuma et al (1990) | E1, E2, L1, L2 | E6, E7 |
| Jeon et al (1995) | E2 | E6, E7 |
| Yoshinouchi et al (1991) | E2 | E6 |

TABLE 2
FREQUENCY OF HPV16 IN CERVICAL CANCERS
BY L1/E6 CONSENSUS PRIMERS COMPARED
TO E6-E7 SPECIFIC PRIMERS

| Reference | Number of sample | L1/E1 | E6/E7* |
|---|---|---|---|
| Van den Brule et al. (1990) | 21 | | 84% |
| Ter Maulen et al. (1992) | 53 | | 38% |
| Guerrero et al. (1992) | 302 | | 48% |
| Prussia et al. (1993) | 20 | | 65% |
| Eluf-Neto et al (1994) | 186 | | 54% |
| Monk et al. (1994) | 218 | | 44% |
| Williamson et al. (1994) | 68 | | 46% |
| Karlsen et al. (1995) | 143 | | 63% |

TABLE 3
VARIABILITY IN RATE OF DETECTION OF
HPV IN CARCINOMA SAMPLES BY L1/E1
CONSENSUS PRIMERS IN DIFFERENT STUDIES

| | | Target and primers | | |
|---|---|---|---|---|
| | | L1 | L1 | E1 |
| Reference | Number of samples | My | Gp | Cp |
| Van den Brule et al. (1990) | 21 | — | 91% | — |
| Ter Maulen et al. (1992) | 53 | — | 89% | — |
| Guerrero et al. (1992) | 302 | 69% | — | — |
| Prussia et al. (1993) | 20 | — | — | 90% |
| Eluf-Neto et al. (1994) | 186 | — | 84% | — |
| Monk et al. (1994) | 218 | 79% | — | — |
| Williamson et al. (1994) | 68 | 81% | — | — |
| Karlsen et al. (1995) | 143 | 91% | 9% | 89% |
| Herrington et al. (1995) | 114 | 54% | — | — |

### BIBLIOGRAPHY

Armstrong B, Holman D. Increasing mortality from cancer of the cervix in young Australian women. Med J Ast 1981; 1:460-62.

Baker C C, Phelps W C, Lindgren V, Braun M J, Gonda M A, Howley P M. Structural and transcriptional analysis of human papillomavirus type 16 sequences in cervical carcinoma cell lines. J Virol 1987; 61:962-71.

Bernard B A, Bailly C, Lenoir M C, Darmon, Thierry F, Yano M. The human papillomavirus type 18 (HPV18) E2 gene product is a repressor of the HPV 18 regulatory region in human keratinocytes. J Virol 1989; 63:4317-4324.

Bosch F X, Manos M M, Munoz M, Sherman M, Jansen A M, Peto J, Schiffinan M H, Moreno V, Jurman R, Shak K V. Prevalence of human papillomavirus in cervical cancer: a worldwide perspective. J Natl Cancer Inst 1995; 87:796-802.

Boshart M, Gissman L, Ikenberg H, Kleinheinz A, Scheurlen W, zur Hausen H. A new type of papillomavirus DNA, its presence in genital cancer biopsies and in cell lines derived from cervical cancer. EMBO J 1984; 3:1151-1157.

Choo K V, Pan C C, Han S H. Integration of human papillomavirus type 16 into cellular DNA of cervical carcinoma: preferential deletion of the E2 gene and invariable retention of the long control region and the E6/E7 open reading frames. Virol 1987; 161:259-61.

Choo K B, Lee H H, Pan C C, Wu S M, Liew L N, Cheung W F, Han S H. Sequence duplication and internal deletion in the integrated human papillomavirus type 16 genome cloned from a cervical carcinoma. J Virol 1988; 62:1659-66.

Cone R W, Minson A C, Smith M R, McDougall J K. Conservation of HPV-16 E6/E7 ORF sequences in a cervical carcinoma. J Med Virol 1992; 37:99-107.

Cullen A P, Reid R, Campion M, Lorinez A T. Analysis of the physical state of different human papillomavirus DNAs in intraepithelial and invasive cervical neoplasms. J Virol 1991; 65:606-612.

Dallas P B, Flanagan J L, Nightingale B N, Morris B J. Polymerase chain reaction for fast, nonradioactive detection of high- and low-risk papillomavirus types in routine cervical specimens and in biopsies. J Med Virol 1989; 27:105-11.

Das B C, Sharma J K, Gopalakrishna V, Luthra U K. Analysis by polymerase chain reaction of the physical state of human papillomavirus type 16 DNA in cervical preneoplastic and neoplastic lesions. J Gen Virol 1992; 73:2327-2336.

de Roda Husman A M, Walboomers J M, van den Brule A J, Meijer C J, Snijders P J. The use of general primers GP5 and GP6 elongated at their 3′ ends with adjacent highly conserved sequences improves human papillomavirus detection by PCR. J Gen Virol 1995; 76(pt 4):1057-62.

Dowhanick J J, McBride A A, Lowly P M. Suppression of cellular proliferation by the papillomavirus E2 protein. J Virol 1995; 69:7791-7799.

Dürst M, Kleinheinz A, Hotz M, Gissman L. The physical state of human papillomavirus type 16 DNA in benign and malignant genital tumours. J Gen Virol 1985; 66(Pt 7):1515-22.

Dürst M, Gallahan D, Jay G, Rhim J S. Glucocorticoid-enhanced neoplastic transformation of human keratinocytes by human papillomavirus type 16 and an activated ras oncogene. Virol 1989; 173(2):767-71.

Eluf-Neto J, Booth M, Munoz N, Bosch F X, Meijer C J, Walboomers J M. Human papillomavirus and invasive cervical cancer in Brazil. Brit J Cancer 1994; 69:114-9.

Figge D C, Bennington J L, Schweid A I. Cervical cancer after initial negative and atypical vaginal cytology. Am J Obstet Gynecol 1970; 108; 422-8.

Fujinaga Y, Shimada M, Okazawa K, Fukushima M, Kato I, Fujinaga K. Simultaneous detection and typing of genital human papillomavirus DNA using the polymerase chain reaction. J Gen Virol 1991; 72(Pt 5): 1039-44.

Fukushima M, Yamakawa Y, Shimano S, Hashimoto M, Sawada Y, Fujinaga K. The physical state of human papillomavirus 16 DNA in cervical carcinoma and cervical intraepithelial neoplasia. Cancer 1990; 66:2155-61.

Gregoire L, Arella M, Campione-Piccardo J, Lancaster W D. Amplification of human papillomavirus DNA sequences by using conserved primers. J Clin Microbiol 1989; 27:2660-5.

Guerrero E, Daniel R W, Bosch F X, Castellsague X, Munoz N, Gili M, Viladiu P, Navarro C, Zubiri M L, Ascunce N, et al. Comparison of ViraPap, Southern hybridization, and polymerase chain reaction methods for human papillomavirus identification in an epidemiological investigation of cervical cancer. J Clin Microbiol 1992; 30:2951-9.

Hildesheim A, Schiffinan M H, Gravitt P E, Glass A G, Greer C E, Zhang T, Scott D R, Rush B B, Lawler P, Sherman M E, Kurman R J, Manos M M. Persistence of type-specific human papillomavirus infection among cytologically normal women. J Inf Dis 1994; 169:235-40.

Howley P M. Presence and expression of human papillomavirus sequences in human cervical carcinoma cell lines. Am J Parthol 1985; 119:361-366.

Jeon S, Allen-Hoffman B L, Lambert P F. Integration of human papillomavirus type 16 into the human genome correlates with a selective growth advantage of cells. J Virol 1995; 69:2989-97.

Karlsen F, Kalantari M, Jenkins A, Pettersen E, Kristensen G, Holm R, Johansson B, Hagmar B. Use of multiple PCR primer sets for optimal detection of human papillomavirus. J Clin Microbiol 1996; 34:2095-100.

Kobayashi Y, Yoshinouchi M, Tianqi G, Nakamura K, Hongo A, Kamimura S, Mizutani Y, Kodama J, Miyagi Y, Kudo T. Presence of human papillomavirus DNA in pelvic lymph nodes can predict unexpected recurrence of cervical cancer in patients with histologically negative lymph nodes. Clin Cancer Res 1998; 4:979-83.

Lorinez A T, Reid R, Jenson A B, Greenberg M D, Lancaster W, Kurman R J. Human papillomavirus infection of the cervix: relative risk association of 15 common anogenital types. Obstet Gynecol 1992; 79:328-37.

Manos M M, Ting Y, Wright D K, Lewis A J, Broker T R, Wolinsky S M. The use of polymerase chain reaction amplification for the detection of genital human papillomaviruses. Cancer Cells 1989; 7:209-14.

Matsukura T, Kanda T, Furuno A, Yoshikawa H, Kawana T, Yoshiike K. Cloning of monomeric human papillomavirus type 16 DNA integrated within cell DNA from a cervical carcinoma. J Virol 1986; 58:979-82.

Matsukura T, Koi S, Sugase M. Both episomal and integrated forms of human papillomavirus type 16 are involved in invasive cervical cancers. Virology 1989; 172:63-72.

Matsukara T, Sugase M. Identification of genital human papillomavirus in cervical biopsy specimens: segregation of specific virus types in specific clinicopathologic lesions. Int J Cancer 1995; 61:13-22.

Morris B J, Flanagan J L, McKinnon K J, Nightingale B N. Papillomavirus screening of cervical lavages by polymerase chain reaction. Lancet 1988; ii(8624):1368.

Morris B J, Nightingale B N. A method of detection of carcinogenic papillomavirus. Australian Patent No. 611135; European Patent No. 88902077.2-2107 (British Patent 0357611, German Patent P 3853678.1, Swiss Patent 3853678.1, Swedish Patent 0357611), U.S. Pat. No. 5,783,412. Priority date Feb. 26, 1987.

Morris B J, Rose B R, Flanagan J L, McKinnon K J, Loo C Y, Thompson C H, Flampoulidou M, Ford R M, Hunter J C, Nightingale B N, Cossart Y E. Automated polymerase chain reaction for papillomavirus screening of cervicovaginal lavages: comparison with dot-blot hybridization in a sexually transmitted diseases clinic population. J Med Virol 1990; 32:22-30.

Munoz N, Bosch F X, de Sanjose S, Tafur L, Izarzugaza I, Gili M, Viladiu P, Navarro C, Martos C, Ascunce N, et al. The causal link between human papillomavirus and invasive cervical cancer: a population-based case-control study in Colombia and Spain. Int J Cancer 1992; 52:743-9.

Noffsinger A E, Suzuk L. Hui Y Z, Gal A A, Fenoglio-Preiser C M. Differential sensitivities of E6 type-specific and L1 consensus primers in the detection of human papillomavirus in anal carcinoma. Mod Pathol 1995; 8(5):509-14.

Park J S, Hwang E S, Park S N, Ahn H K, Um S J, Kim C J, Kim S J, Namkoong S E. Physical status and expression of HPV genes in cervical cancers. Gynecol Oncol 1997; 65:121-9.

Pfister H, Fuchs P G. Relation of papillomaviruses to anogenital cancer. Dermatol Clin 1991; 9:267-76.

Romanezuk H, Thierry F, Howley P M. Mutational analysis of cis elements involved in E2 modulation of human papillomavirus type 16 P99 and type 18 P105 promoters. J Virol 1990; 64(6):5240-9.

Schneider-Maunoury S, Croissant O, Orth G. Integration of human papillomavirus type 16 DNA sequences: a possible early event in the progression of genital tumours. J Virol 1987; 61:3295-8.

Schwarz E, Freese U, Gissmann L, Mayer W, Roggenbuck B, Stremlau A, zur Hausen H. Structure and transcription of human papillomavirus sequences in cervical carcinoma cells. Nature 1985; 314:111-4.

Shirasawa H, Tomita Y, Kubota K, Kasai T, Sckiya S, Takamizawa H, Simizu B. Detection of human papillomavirus type 16 DNA and evidence for integration into the cell DNA in cervical dysplasia. J Gen Virol 1986; 67:2011-5.

Shirasawa H, Tomita Y, Sckiya S, Takamizawa H, Simizu B. Integration and transcription of human papillomavirus type 16 and 18 sequences in cell lines derived from cervical carcinomas. J Gen Virol 1987; 68:583-91.

Smits H L, Bollen L J, Tjong-A-Hung S P, Vonk J, Van der Velden J, Ten Kate F J, Kaan J A, Mol B W, Ter Schegget J. Intermethod variation in detection of human papillomavirus DNA in cervical smears. J Clin Microbiol 1995; 33:2631-6.

Snijders P K, van den Brule A J, Schrijnemakers H F, Snow G, Meijer C J, Walboomers J M. The use of general primers in the polymerase chain reaction permits the detection of a broad spectrum of human papillomavirus genotypes. J Gen Virol 1990; 71:173-81.

Stoler M H, Rhodes C R, Whitbeck A, Wolinsky S M, Chow L T, Broker T R. Human papillomavirus type 16 and 18 gene expression in cervical neoplasias. Human Pathol 1992; 23:117-28.

Thierry F, Howley P M. Functional analysis of E2 mediated repression of the HPV 18 P105 promoter. New Biol 1991; 3:90-100.

Tsunokawa Y, Takebe N, Nozawa S, Kasamatsu J, Gissman L, zur Hausen H, Terada M, Sugimura T. Presence of human papillomavirus type-16 and type-18 DNA sequences and their expression in cervical cancers and cell lines from Japanese patients. Int J Cancer 1986; 37:499-503.

Turek. Adv. Virus Res. 44:305-356 (1994).

Vernon D D, Unger E R, Miller D L, Lee D R, Reeves W C. Association of human papillomavirus type 16 integration in the E2 gene with poor disease-free survival from cervical cancer. Int J Cancer 1997; 74:50-56.

Wagatsuma M, Hashimoto K, Matsukura T. Analysis of integrated human papillomavirus type 16 DNA in cervical cancers: amplification of viral sequences together with cellular flanking sequences. J Virol 1990; 64:813-21.

Wei L, Dai-Shu H, Ju Y, Andrieu J-M. Nature 1994, 368:269-271.

Yee C, Krishnan-Hewlett I, Backer C C, Schlegal R, Howley P M. Presence and expression of human papillomavirus sequences in human cervical carcinoma cell lines. Am J Pathol 1985; 119:361-6.

Yiu K C, Huang D P, Chan M K, Foo W. The physical state of human papillomavirus type 16 DNA in cervical carcinomas of Hong Kong Chinese. Oncogene1991; 6:1339-42.

1. A method for determining the progression of human papillomavirus infection which comprises detecting the presence or absence of a E6, E7 or E6/E7 region of the human papillomavirus and the presence or absence of a L1, L2 or L1/L2 region of the human papillomavirus wherein the absence of the E6, E7 or E6/E7 region and the absence of the L1, L2 or L1/L2 region signifies no human papillomavirus infection; the presence of the E6, E7 or E6/E7 region and the presence of the L1, L2 or L1/L2 region signifies an early stage human papillomavirus infection and the presence of the E6, E7 or E6/E7 region and the absence of the L1, L2 or L1/L2 region signifies a late stage human papillomavirus infection.

2. The method according to claim 1, wherein polymerase chain reaction is used to detect the presence or absence of the E6, E7 or E6/E7 region and the presence or absence of the L1, L2 or L1/L2 region.

3. The method according to claim 1, wherein polymerase chain reaction-enzyme linked immunosorbent assay is used to detect the presence or absence of the E6, E7 or E6/E7 region and the presence or absence of the L1, L2 or L1/L2 region.

4. The method according to claim 1, wherein ligase chain reaction is used to detect the presence or absence of the E6, E7 or E6/E7 region and the presence or absence of the L1, L2 or L1/L2 region.

5. The method according to claim 1, wherein Q-beta replicase amplification is used to detect the presence or absence of the E6, E7 or E6/E7 region and the presence or absence of the L1, L2 or L1/L2 region.

6. The method according to claim 1, wherein PCR and Taq man are used to detect the presence or absence of the E6, E7 or E6/E7 region and the presence or absence of the L1, L2 or L1/L2 region.

7. The method according to claim 1, wherein nucleic acid sequence based amplification is used to detect the presence or absence of the E6, E7 or E6/E7 region and the presence or absence of the L1, L2 or L1/L2 region

8. The method according to claim 1, wherein the presence or absence of the E6, E7 or E6/E7 region and the presence or absence of the L1, L2 or L1/L2 region is determined by detecting the presence or absence of DNA from the E6, E7 or E6/E7 region and the presence or absence of DNA from the L1, L2 or L1/L2 region.

9. The method according to claim 1, wherein the presence or absence of the E6, E7 or E6/E7 region and the presence or absence of the L1, L2 or L1/L2 region is determined by detecting the presence or absence of RNA coded by DNA from the E6, E7 or E6/E7 region and the presence or absence of RNA coded by DNA from the L1, L2 or L1/L2 region.

10. The method according to claim 1, wherein the presence or absence of the E6, E7 or E6/E7 region and the presence or absence of the L1, L2 or L1/L2 region is determined by detecting expression of the E6, E7 or E6/E7 region and the presence or absence of expression of the L1, L2 or L1/L2 region.

11. The method according to claim 1, wherein the human papillomavirus is selected from the group consisting of 1a, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 47, 48, 49, 50, 51, 52, 53, 54, 55, 56, 57, 58, 59, 60, 61, 62, 63, 64, 65, 66, 67, 68, 69, 70, 72, 73, 74, 75, 76, and 77.

12. The method according to claim 1, wherein the human papillomavirus is selected from the group consisting of human papillomavirus 6, 11, 16, 18, 31, 33, 35, 39, 41, 42, 43, 44, 49, 50, 52, 54, 55, 56, and 68a.

13. The method according to claim 1, wherein the human papillomavirus is selected from the group consisting of human papillomavirus 6, 11, 16, 18 and 33.
